(19) United States
(12) Patent Application Publication
(10) Pub. No.: US 2015/0306559 A1
(43) Pub. Date: Oct. 29, 2015

(54) BIOMASS PROCESSING SYSTEM AND PROCESSING METHOD

(71) Applicant: TSUKISHIMA KIKAI CO., LTD., Tokyo (JP)

(72) Inventor: Tomoki HAYAKAWA, Tokyo (JP)

(21) Appl. No.: 14/409,380

(22) PCT Filed: Mar. 27, 2013
(86) PCT No.: PCT/JP2013/058946

§ 371 Date: Dec. 18, 2014

(30) Foreign Application Priority Data
Jun. 21, 2012 (JP) 2012-140021

(51) Int. Cl. B01J 19/00; C12M 1/34; C12M 1/40; B01J 19/24

(52) U.S. Cl. CPC B01J 19/0006; B01J 19/24; B01J 19/0013; C12M 41/00; C12M 21/18; B01J 2219/00056; B01J 2219/00177; B01J 2219/00049; B01J 2219/24

(57) ABSTRACT

A system and a method by which the high yield of a final product produced through a pretreatment and a saccharification and fermentation step can be stably obtained, even if a property of the biomass fed to a pretreatment device or the amount of a contained acid component in the biomass may be variable. The biomass is pretreated by a pretreatment device. At least one of pH, acidity, alkalinity, and ultraviolet absorbance of the pretreated biomass is measured by a measurement unit. The amount of a generated decomposition product or a generated undecomposed substance in the pretreatment is obtained on the basis of a measurement result, and an operation condition of the pretreatment device or a property of the biomass before the pretreatment are controlled on the basis of the calculated result.

### TECHNICAL FIELD

The present invention relates to a biomass treatment system and a method for treating biomass, which is pretreated by a hydrothermal treatment, a thermal treatment, an acid/alkali treatment, and the like, in particular, relates to a biomass treatment system and a method for treating biomass, in which the amount of an undecomposed substance after a pretreatment is optimized by controlling an operation condition of a pretreatment device or a property of the biomass before the pretreatment.

### BACKGROUND ART

The biomass containing cellulose and hemicellulose of bagasse, wheat straw, rice straw, a palm residue, switchgrass, paper, and the like has been mainly used as animal feed, and as recent advances in a cellulose saccharification technique, an application where the biomass is used as a raw material of ethanol or an organic acid is gradually being developed. In producing the ethanol or the organic acid from the biomass containing the cellulose and hemicellulose, a fibrous material such as the cellulose and hemicellulose, which is a main component of the biomass, is subjected to a pretreatment such as a hydrothermal treatment and the like; the pretreated biomass is subjected to sterilization; the sterilized biomass is reacted with a saccharifying enzyme to obtain a saccharified solution containing saccharides such as glucose, xylose, arabinose, galactose, and mannose; and the saccharides are converted to the ethanol or the organic acid by a fermentation method using a microorganism such as yeast. In addition, is also frequently used a technique in which enzymatic saccharification and fermentation are performed at the same time in a simultaneous saccharification and fermentation tank for the biomass after the pretreatment and the sterilization.

The biomass is a composite, in which mainly three components of cellulose, hemicellulose, and lignin are firmly bound, and further a cellulose molecule itself has a hard crystal structure, therefore, without any treatment, a cellulase as a saccharifying enzyme cannot approach to the cellulose molecule. It is required thus that the biomass is pretreated by any of a thermal treatment, a hydrothermal treatment, an acid treatment, an alkali treatment, milling, wood rotting fungi, and the like in a pretreatment device so as to loosen binding of the three components, resulting in that the biomass is converted so as to have easily saccharified configuration and properties.

It is preferable that the yield of the ethanol and the organic acid, which are finally obtained, is stable, however, even if the kind and the charged amount of the biomass to be fed to the pretreatment device, and the operation condition of the pretreatment device is maintained constant, the yield of a final product such as the ethanol may fluctuate. Factors that generate the variation will be explained by way of an example of the bagasse used as the biomass as follows. (1) Since the property of the bagasse changes depending on the harvest season of sugarcane, the sugar production process thereof, the storage method and the storage period of the bagasse, and the like, the difference is generated in sensitivity to the pretreatment. (2) Further, as another factor, there may be the following case. Sugar sticks to the bagasse in the process of squeezing the sugarcane and when such sugar is left without any treatment, an acid component is generated by lactic acid fermentation. Then, the amount of the stuck sugar differs depending on the kind of the bagasse, therefore the amount of the generated acid component also differs depending thereon. Accordingly, a degree of progress in the pretreatment step may vary depending on the amount of the acid component. Precisely, since variation in the degree of the progress in the pretreatment step is caused by the factors stated in (1) and (2), the amount of by-product derived from the pretreatment is changed.

Patent Literatures 1 to 5 listed below can be raised as the prior art documents relating to the present invention. In Patent Literature 1, there is a disclosure of an invention in which amorphous chitin or amorphous chitosan is decomposed under a certain hydrothermal condition to low molecules, and further there is a disclosure that the decomposition is controlled by the reaction time or the reaction temperature. Then, in Patent Literature 2, there is a disclosure that when an organic substance is decomposed by a hydrothermal reaction, the amount of CO contained in a processing fluid is measured, and the hydrothermal reaction is controlled so that the measured amount of CO is the predetermined value or less. Further, in Patent Literature 3, there is a disclosure that an organic halide is decomposed by an organic halide decomposition device, distribution of the organic halide and/or a decomposition product concentration thereof in waste water is measured by a laser ionization time of flight mass spectrometer, and a decomposition treatment condition for the organic halide is controlled under an optimal condition on the basis of the measured distribution. In addition, in Patent Literature 4, there is a disclosure that a high aqueous organic substance is decomposed in a hydrothermal reaction with microwaves while a property of the organic substance is analyzed, and output of the microwaves is controlled on the basis of the analysis result. Further, in Patent Literature 5, there is a disclosure of a method of producing favorable liquid animal feed from proteins hydrolyzed in a high temperature and high pressure water treatment, and there is a description that it was found out that the yield of a target amino acid might be increased by appropriately controlling a reaction condition of pressure or the like.

### CITATION LIST

### Patent Literature

Patent Literature 1: Japanese Patent Application Laid-Open No. 2003-212902

Patent Literature 2: Japanese Patent Application Laid-Open No. 2003-181406

Patent Literature 3: Japanese Patent Application Laid-Open No. 2002-360727

Patent Literature 4: Japanese Patent Application Laid-Open No. 2006-095475

Patent Literature 5: Japanese Patent Application Laid-Open No. 2008-167748

### SUMMARY OF INVENTION

### Technical Problem

Since the by-product has an effect of inhibiting growth and metabolism of fermentive microorganisms, when the by-product is generated in a large amount by the pretreatment, the amount of the final product is decreased. In addition, the amount of generated saccharides by enzymatic saccharification changes depending on the degree of the progress in the pretreatment. As described above, even if the kind and the charged amount of the biomass fed to the pretreatment device, and the operation condition of the pretreatment device are maintained constant, the degree of the progress in the pretreatment and the amount of the generated by-product fluctuate, therefore, there is a problem that high yield of the final product cannot be obtained stably.

A main object of the present invention is, therefore, to stably obtain the high yield of the final product through the pretreatment and the saccharification and fermentation step even if there is a difference in a property of biomass fed to the pretreatment device or in the amount of the acid component contained in the biomass.

### Solution to Problem

The present invention to solve the above problems is as follows.

### Invention According to Claim 1

A biomass treatment system, comprising:

a pretreatment device pretreating a biomass; a measurement unit measuring at least one selecting from

the group consisting of pH, acidity, alkalinity, ultraviolet absorbance, an amount of a decomposition product, and an amount of an undecomposed substance of the pretreated biomass; and

a control unit controlling an operation condition of the pretreatment device or a property of the biomass before the pretreatment on the basis of a measurement result obtained by the measurement unit.

### Operation and Effect

The amount of the decomposition product or the undecomposed substance generated by the pretreatment, the pH, the acidity, the alkalinity, or the ultraviolet absorbance of the pretreated biomass is used as an index of a degree of progress in a pretreatment reaction, the operation condition of the pretreatment device or the property of the biomass before the pretreatment is controlled on the basis of the measurement result.

The yield of saccharides in an enzymatic saccharification step differs depending on the degree of the progress in the pretreatment. Therefore, by an appropriate management of the progress in the pretreatment reaction using the amount of the decomposition product or the like as the index, the yield of the saccharides in the enzymatic saccharification step can be stabilized.

In addition, in the decomposition product obtained by the pretreatment, an inhibitant is contained, which inhibits the progress in a fermentation step performed later, however, by the appropriate management of the progress in the pretreatment reaction, the amount of the generated inhibitant can be decreased, and the yield in the fermentation step can be stabilized.

Accordingly, the high yield of a final product produced through the enzymatic saccharification step and the fermentation step can be stably obtained.

Further even in simultaneous saccharification and fermentation in which the enzymatic saccharification and the fermentation are performed in the same tank, the same effect can be obtained.

### Invention According to Claim 2

The biomass treatment system according to claim 1,

wherein the control unit controls at least one of reaction time of the pretreatment, a reaction temperature of the pretreatment, the pH of the biomass before the pretreatment, the acidity of the biomass before the pretreatment, and the alkalinity of the biomass before the pretreatment.

### Operation and Effect

By controlling of the reaction time and the reaction temperature of the pretreatment, the amount of the generated inhibitant can be decreased, and further the degree of the progress in the pretreatment can be made in an appropriate state.

In addition, by the control of at least one of the pH, the acidity, and the alkalinity of the biomass before the pretreatment, the amount of an acid component contained in the biomass before the pretreatment becomes appropriate, therefore, the amount of the generated inhibitant can be decreased, and further the degree of the progress in the pretreatment can be made in the appropriate state.

### Invention According to Claim 3

The biomass treatment system according to Claim 1 or 2, further comprising:

a saccharification and fermentation unit in which the biomass pretreated under controlling by the control unit is saccharified with a saccharifying enzyme, and the saccharified biomass is fermented with a fermentive microorganism.

### Operation and Effect

The pretreated biomass that has been generated under the appropriately controlled pretreatment has the less content of the inhibitant, which inhibits the progress in the fermentation step, and further the binding of the three components of the cellulose, the hemicellulose, and the lignin in the biomass is sufficiently loosened so that the saccharifying enzyme can approach easily to the cellulose molecule, therefore, the high yield of the final product produced through the enzymatic saccharification step and the fermentation step can be stably obtained.

### Invention According to Claim 4

The biomass treatment system according to claim 1 or 2, further comprising:

a saccharification and fermentation unit in which the biomass pretreated under controlling by the control unit is saccharified with a saccharifying enzyme, and the saccharified biomass is fermented with a fermentive microorganism;

a saccharifying enzyme production device producing the saccharifying enzyme to be used in the saccharification and fermentation unit;

a first treatment unit sending apart of the pretreated biomass without any treatment to the saccharification and fermentation unit; and

a second treatment unit feeding a rest of the pretreated biomass to the saccharifying enzyme production device, propagating a microorganism which produces the saccharifying enzyme by using the fed pretreated biomass as a medium so as to produce the saccharifying enzyme, and sending the produced saccharifying enzyme to the saccharification and fermentation unit.

### Operation and Effect

The pretreated biomass that has been generated under the appropriately controlled pretreatment has the less content of the inhibitant, which inhibits the progress in a saccharifying enzyme production step, therefore, the high yield of the saccharifying enzyme produced through the saccharifying enzyme production step can be stably obtained.

### Invention According to Claim 5

A method for treating a biomass, comprising:

a step of pretreating the biomass by a pretreatment device;

a step of measuring at least one selecting from the group consisting of pH, acidity, alkalinity, ultraviolet absorbance, an amount of a decomposition product, and an amount of an undecomposed substance of the pretreated biomass by a measurement unit; and

a step of controlling an operation condition of the pretreatment device or a property of the biomass before the pretreatment on the basis of a measurement result obtained by the measurement unit.

### Operation and Effect

The same action effect as in claim 1 is exerted.

### Advantageous Effects of Invention

According to the present invention, in spite of differences in the property of the biomass to be fed to the pretreatment device or in the amount of the acid component contained in the biomass, the high yield of the final product obtained through the pretreatment and the saccharification and fermentation step can be stably obtained.

### BRIEF DESCRIPTION OF DRAWINGS

FIG. 1 is the flow chart of the biomass treatment according to the present invention.

FIG. 2 is the graph showing the relationship between the amount of the residual solid and the ethanol yield.

FIG. 3 is the graph showing the relationship between the ultraviolet absorbance and the ethanol yield.

FIG. 4 is the graph showing the relationship between the acidity and the ethanol yield.

FIG. 5 is the graph showing the relationship between the pH and the ethanol yield.

### DESCRIPTION OF EMBODIMENTS

### Biomass Treatment Step

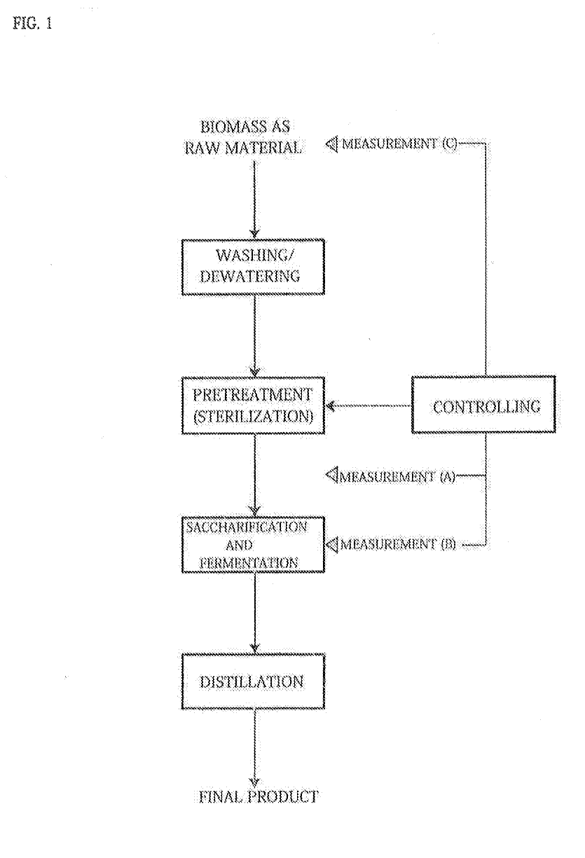

FIG. 1 shows the schematic flow diagram of the biomass treatment according to the present invention.

A system according to the present invention can suitably be used for biomass containing cellulose and hemicellulose of bagasse, wheat straw, a palm residue, corn stover, a palm coconut residue, a cassava residue, woodchips, wastewood, jute, kenaf, switchgrass, used paper, and the like.

### Washing and Dewatering Step

The biomass containing the cellulose and the hemicellulose is separated from foreign materials such as sand, and stones by using water in a washing step, and a large part of the water added in the washing step is removed in a dewatering step.

### Pretreatment and Sterilization Step

The biomass is a composite, in which mainly three components of cellulose, hemicellulose, and lignin are firmly bound, and further a cellulose molecule itself has a hard crystal structure, therefore, without any treatment, the cellulase as a saccharifying enzyme cannot approach to the cellulose molecule.

It is required thus that the biomass after dewatering is pretreated by any of a thermal treatment, a hydrothermal treatment, an acid treatment, an alkali treatment, milling, wood rotting fungi, and the like in the pretreatment device so as to loosen binding of the three components. After that, sterilization is generally performed in order to prevent contamination. However, when the pretreatment of any of the thermal treatment, the hydrothermal treatment (steaming, steam explosion), the acid treatment (a dilute acid process and a concentrated acid process using sulfuric acid, nitric acid, phosphoric acid, and the like, or a SO2 impregnated steam explosion process), and an alkali treatment (a caustic soda process, a sodium sulfite process, an ammonia process, or a calcium hydroxide process) is performed, the pretreatment has a sterilization effect. That is to say, this has an advantage that the sterilization step is not required to be performed separately from the pretreatment step. In a case of a concentrated sulfuric acid method, the pretreatment and the saccharification can be performed without using a cellulase.

### Saccharification Step

The pretreated biomass is sent to a saccharification device and saccharified in the saccharification device. A typical saccharification device is a bioreactor. In the saccharification device, the pretreated biomass is decomposed mainly by the action of three kinds of cellulase (endoglucanase (EG), cellobiohydrolase (CBH), and β-glucosidase (BGL)). The cellulase is added in the amount of 3 to 30 FPU/g, and preferably 5 to 20 FPU/g of the dry weight of the biomass as a raw material (FPU/g is a unit of enzymatic activity of the cellulase which produces 10.8 mg of glucose on filter paper for 60 minutes).

### Fermentation Step

The saccahrified material is sent to a fermentation device, and fermented in the fermentation device. In order to convert saccharides to the ethanol, yeast of Saccharomyces, Schizosaccharomyces, Kluyveromyces, Pichia, or Candida; a bacterium of Zymomonas, or Clostridium; or a genetically modified microorganism induced with a specific gene thereof can be used. The ethanol fermentative bacterium is inoculated, for example, in a case of Saccharomyces cerevisiae, in an amount of 1 to 100 g wet-weight/L, and preferably 5 to 50 g wet-weight/L-volume of fermentation broth. In addition, in order to convert the saccharides to lactic acid, for example, a filamentous fungus such as Rhizopus oryzae can be used. Further, in order to convert the saccharides to succinic acid, for example, a coryneform bacterium, Bacillus, Rhizobium, or the like can be used.

### Simultaneous Saccharification and Fermentation Step

In the above, a case where the saccharification and the fermentation are performed separately in different devices was shown, however, the saccharification and the fermentation maybe performed at the same time in one device.

When biomass is saccharified (hydrolyzed) with a saccharifying enzyme, there is a problem that enzymatic activity is inhibited by accumulation of the generated saccahrified material, and the saccharification yield thereof is lowered. However, in the simultaneous saccharification and fermentation, the saccharification with a saccharifying enzyme and the fermentation of the saccharified material with fermentive microorganisms can be performed at the same time. Accordingly, the enzymatic activity is rarely inhibited by the accumulation of the generated saccharified material, because before that, fermentative bacterium such as the yeast converts the saccharides, resulting in an advantage that the enzymatic activity can be maintained. Therefore, the saccharification and the fermentation are more preferably performed at the same time in one device than being performed separately in different devices.

### Evaporation Step

The fermentation broth generated by the fermentation is sent to a mash column or an evaporator. In the mash column, the fermentation broth is heated to the temperature of around the boiling point, and the evaporated ethanol or the evaporated organic acid is condensed to obtain a high concentration ethanol- containing liquid or a high concentration organic acid-containing liquid. The concentration of the ethanol or the organic acid is around 30% or less, and more specifically around 3 to 20%. On the other hand, a liquid containing a component having a boiling point higher than that of the ethanol or the organic acid, or a solid such as cellulose or lignin for which the saccharification and fermentation has not been performed, is discharged from the bottom of the mash column. In addition, the same operation as in the above is performed also in the evaporator.

### Rectification Step

The ethanol-containing liquid or organic acid-containing liquid obtained through the evaporation step is sent to a rectification column or a distillation column. In the rectification column, the ethanol-containing liquid or the organic acid-containing liquid is heated to the temperature in the vicinity of the boiling point of a target component, and the evaporated ethanol or the organic acid are condensed to obtain a high concentration ethanol-containing liquid or an organic acid-containing liquid. In a case where the product is ethanol, the ethanol concentration in a fraction from the rectification column is 90 to 95 volume %. On the other hand, wastewater generated in the rectification step is discharged from the bottom of the rectification column. The same operation as in the above is performed also in the distillation column.

In order to further increase the concentration or to remove impurities, there may be a case where another rectification step is further provided, or a dewatering step is provided, depending on the required quality of the final product.

### Measurement and Control Step

When the biomass containing the cellulose and hemicellulose is pretreated, a part of the hemicellulose is mainly decomposed and liquefied. Since the amount of the decomposition products is increased due to a strong condition of the pretreatment, the progress of the pretreatment is determined on the basis of the amount of a decomposition product in the present invention. In the same manner, the progress of the pretreatment may be determined on the basis of the amount of undecomposed residual solid.

When the progress of the pretreatment is insufficient, since the saccharifying enzyme, cellulase cannot sufficiently approach to the cellulose molecule, the yield of the target substance is lowered in the saccharification and fermentation step performed later. Therefore, the degree of the progress in the pretreatment is detected with reference to the amount of the generated decomposition product or the amount of the residual solid as the index, and the operation condition of the pretreatment device or the property of the biomass before the pretreatment are controlled so as to stably obtain the final product with high yield that is produced through the saccharification and fermentation step.

In addition, since the decomposition product obtained by the pretreatment contains the inhibitant, which inhibits the progress in the fermentation step performed later, when the amount of the generated inhibitant is increased excessively due to the excessive progress of the pretreatment, the amount of the final product obtained through the fermentation step is decreased. Therefore, the degree of the progress in the pretreatment is detected with reference to the amount of the generated decomposition product or the amount of the residual solid as the index, and the operation condition of the pretreatment device or the property of the biomass before the pretreatment are controlled so as to appropriately control the progress of the pretreatment reaction. Thus, the amount of the generated inhibitant can be decreased, and the final product produced through the saccharification and fermentation step can be stably obtained at high yield.

Further, the pH, the acidity, the alkalinity, or the ultraviolet absorbance of the pretreated biomass is measured, and the progress of the pretreatment can also be determined based on the measurement result. In addition, by controlling of the operation condition of the pretreatment device or the property of the biomass before the pretreatment with reflecting the degree of the progress in the pretreatment, the final product produced through the saccharification and fermentation step can be stably obtained at high yield.

The foregoing will be specifically described by way of specific examples.

First, during transporting of pretreated biomass from the pretreatment device to the saccharification and fermentation device (for example, bioreactor), the measurement (A) of the pretreated biomass is performed. Specifically, at least one of (1) the measurement of the acidity or the alkalinity by an automatic titrator, (2) the measurement of the pH by a pH sensor, or (3) the measurement of the ultraviolet absorbance by a UV-Vis absorption photometer, is performed. The measurements of (1) to (3) are all performed for the biomass in a fluid state. More specifically, the measurements of (1) and (2) can be performed for slurry, while in the measurement of (3) the pretreated biomass should be separated into a solid component and a liquid component for measuring the separated liquid component.

Due to the high concentration of the solid component in the pretreated biomass during the transportation, there may be a case where the measurement of the pretreated biomass is difficult without any treatment. In that case, the pretreated biomass is mixed with the large amount of water, into which the liquid component of the pretreated biomass is extracted, is applied to each of the measurement means.

Alternatively, in place of the measurement (A) or with the measurement (A), the measurement (B) of an internal liquid of the bioreactor may be performed. Since the internal liquid of the bioreactor is in a liquid state, the measurement can be performed without the extraction.

The degree of the progress in the pretreatment is detected on the basis of the measurement result of the pH, the acidity, the alkalinity, or the ultraviolet absorbance of the pretreated biomass. For example, in the treatment system according to the present invention, a memory device is provided, in which statistical data is stored, related to a relationship between each value of the pH, the acidity, the alkalinity or the ultraviolet absorbance of the pretreated biomass and the degree of the progress in the pretreatment. Then, the statistical data in the memory device is compared to the measurement result so as to detect the degree of the progress in the pretreatment.

In the present invention, the ultraviolet absorbance of the pretreated biomass at 280 nm is preferably in the range of 200 to 450. If the ultraviolet absorbance is lower than 200, the pretreatment condition would be strong, therefore, the amount of the generated inhibitant would be increased and the fermentation step would be inhibited, as a result, the yield of the final product would be lowered. On the other hand, if the ultraviolet absorbance is higher than 450, the pretreatment condition would be weak, therefore, the saccharifying enzyme, the cellulase could not sufficiently approach to the cellulose molecule, as a result, the yield of the final product produced through the saccharification and fermentation step performed later would be lowered.

Further, in the present invention, the acidity of the pretreated biomass is preferably in the range of 20 to 45 [g-as acetic acid/kg-solid in the pretreated material]. If the acidity is lower than 20 [g-as acetic acid/kg-solid in the pretreated material], the pretreatment condition would be weak, therefore, the saccharifying enzyme, cellulase could not sufficiently approach to the cellulose molecule, as a result, the yield of the final product produced through the saccharification and fermentation step performed later would be lowered. On the other hand, if the acidity is higher than 45 [g-as acetic acid/kg-solid in the pretreated material], the pretreatment condition would be strong, therefore, the amount of the generated inhibitant would be increased and the fermentation step would be inhibited, as a result, the yield of the final product would be lowered.

In the present invention, the pH of the pretreated biomass is preferably in the range of 3.2 to 3.7. If the pH is lower than 3.2, the pretreatment condition would be strong, therefore, the amount of the generated inhibitant would be increased and the fermentation step would be inhibited, as a result, the yield of the final product would be lowered. On the other hand, if the pH is higher than 3.7, the pretreatment condition would be weak, therefore, the saccharifying enzyme, cellulase could not sufficiently approach to the cellulose molecule, as a result, the yield of the final product produced through the saccharification and fermentation step performed later would be lowered.

When the measurement result of the pH, the acidity, the alkalinity, or the ultraviolet absorbance of the pretreated biomass is not in the respective range described above, the degree of progress in the pretreatment is determined to be not preferable, the operation condition of the pretreatment device or the property of the biomass before the pretreatment are controlled so that the pretreatment can progress appropriately. Specifically, when the pH, the acidity, the alkalinity, or the ultraviolet absorbance is not in the respective range described above, the amount of added alkali content to the biomass before the pretreatment, the amount of the added acid content thereto, the reaction time of the pretreatment device, or the reaction temperature thereof is adjusted so that the measurement result is in the respective range stated above.

More specifically, in the pretreatment, longer the time or higher the temperature, the reaction is held in more severe condition so that the reaction progresses further and the generated inhibitant increases. Further as the condition of the reaction becomes severe, the amount of the residual solid is decreased, the ultraviolet absorbance (280 nm) is increased, the acidity is increased, and the pH is lowered. Therefore, with reference to the measurement results, these reaction conditions can be adjusted so as to fall within the appropriate ranges, respectively.

Out of the pH, the acidity, the alkalinity, and the ultraviolet absorbance of the pretreated biomass, it is enough to control any one of these reaction conditions according to the measurement results thereof. However, it is more preferable to control two or more reaction conditions according to the measurement results thereof. By doing so, the degree of the progress in the pretreatment can be controlled more reliably.

Alternatively, in place of the detecting stated above, the amount of the residual solid in the pretreatment maybe measured. In this case, the amount of the solid in the biomass to be fed to the pretreatment step and the amount of solid in the biomass after the pretreatment are measured. Specifically, the weight of the biomass to be fed to the pretreatment step is measured by a metric conveyor or the like (weight W1 (ton)), and the water content is measured by an infrared moisture analyzer or the like (water content M1 (%)). By using similar equipments, the weight of the biomass after the pretreatment (W2 (ton)) and the water content (M2 (%)) are measured.

Further, according to the following calculus equation, the amount of the residual solid W3 (kg) derived from 1 ton of the fed biomass solid is obtained.

W3 (kg)=(W2×(100−M2 (%))×1000/(W1×(100−M1 (%)) [Mathematical 1]

The degree of the progress in the pretreatment is detected on the basis of the calculated amount of the residual solid. For example, in the treatment system according to the present invention, the memory is provided, in which statistical data is stored, related to the relationship between each value of the amount of the residual solid and the degree of the progress in the pretreatment. Then, the statistical data in the memory is compared to the measurement result so as to detect the degree of the progress in the pretreatment.

In the present invention, the amount of the residual solid is preferably in the range of 60 to 75% of the amount of the fed biomass. In other words, when 1 ton of the biomass is fed to the pretreatment device, the amount of the residual solid after the pretreatment is preferably in the range of 600 to 750 kg. If the amount of the residual solid for 1 ton of the fed biomass is less than 600 kg, the condition of the pretreatment would be strong, therefore, the amount of the generated inhibitant would-be increased so as to inhibit the fermentation step, as a result, the yield of the final products would be lowered. On the other hand, if the amount of the residual solid is larger than 750 kg, the condition of the pretreatment would be weak, therefore, the saccharifying enzyme, cellulase could not sufficiently approach to the cellulose molecule, as a result, the yield of the final products produced through the saccharification and fermentation step performed later would be lowered.

When the amount of the residual solid is not in the range described above, the degree of progress in the pretreatment is determined to be not preferable, the operation condition of the pretreatment device is controlled so that the pretreatment can progress appropriately. Specifically, when the amount of the residual solid for 1 ton of the fed biomass is less than 600 kg, the reaction time is controlled so as to be shorter or the reaction temperature is controlled so as to be lower in order to make the condition of the pretreatment weak. On the other hand, when the amount of the residual solid is larger than 750 kg, the reaction time is controlled so as to be longer or the reaction temperature is controlled so as to be higher in order to make the condition of the pretreatment strong.

In the above explanation, it is exemplified that the controlling is performed on the basis of the amount of the residual solid. Alternatively, the controlling may be performed on the basis of the amount of the decomposition product. In this case, the amount of the decomposition product is preferably controlled so as to fall within the range of 25 to 40% of the amount of the fed biomass. If the amount of the decomposition product is higher than 40%, the condition of the pretreatment would be strong, therefore, the amount of the generated inhibitant would be increased so as to inhibit the fermentation step, as a result, the yield of the final product would be lowered. On the other hand, if the amount of the residual solid is lower than 25%, the condition of the pretreatment would be weak, therefore, the saccharifying enzyme, cellulase could not sufficiently approach to the cellulose molecule, as a result, the yield of the final product produced through the saccharification and fermentation step performed later would be lowered.

In the foregoing explanation, the values of the pH, the acidity, the alkalinity, the ultraviolet absorbance, the amount of the residual solid, and the amount of the decomposition product that define the appropriate ranges are obtained as the results of experiments stated after. However, the present invention is not limited to these values, it is possible to set other desired ranges of values and control the operation condition of the pretreatment device or the property of the biomass before the pretreatment so as to fall within the set desired ranges, respectively.

### Saccharifying Enzyme Production Step

In the present invention, the pretreated biomass generated under the appropriately controlled pretreatment may be used in the saccharifying enzyme production step. The step is performed by the saccharifying enzyme production device. The saccharifying enzyme production step comprises a step propagating an enzyme-producing microorganism and a step producing an enzyme with the propagated enzyme-producing microorganism, as explained in detail below.

At first, a useful strain is prepared for producing the target enzyme. The useful strain to be prepared includes for example a cellulase-producing microorganism for producing the cellulase, and a hemicellulase-producing microorganism for producing the hemicellulase. More specifically, anyone or more microorganisms of a fungus of Acremonium, Trichoderma, Penicillium, Aspergillus, and Thermoascus, and a eubacterium of Clostridium and Bacillus can be used.

As the microorganism which produces the saccharifying enzyme, a fungus belonging to Acremonium cellulolyticus is preferably used. In a case where the enzyme-producing microorganism is Acremonium cellulolyticus, when the same pretreated biomass is used as the raw material for the saccharification and for the enzyme production, the yield of the saccharides in the saccharification step can be increased due to the particularly large amount of produced hemicellulase. The fungus belonging to Acremonium cellulolyticus includes specifically an Acremonium cellulolyticus Y-94 strain, an Acremonium cellulolyticus T-N strain, an Acremonium cellulolyticus C-1 strain, and an Acremonium cellulolyticusCF-2612 strain. Among of them, the Acremonium cellulolyticus C-1 strain and the Acremonium cellulolyticus CF-2612 strain are particularly preferably used due to their high xylanase activity.

Next, the prepared useful strain is propagated in a medium. As the medium, a solid medium, a semifluid medium, and a liquid medium (bouillon, broth) can be used and the liquid medium is preferably used. Further, an organic nutrient may be contained in the medium as needed.

In general, for the medium, high purity cellulose, monosaccharides, and disaccharides are frequently used as a carbon source. However, since the high purity cellulose, the monosaccharides, and the disaccharides are expensive resulting in high production cost, instead of them, the pretreated biomass can be used. More specifically, by the pretreatment of lignocellulosic biomass, the pretreated biomass containing the cellulose can be generated. Subsequently, the rest of the pretreated biomass is sent to the saccharifying enzyme production device by using a second treatment unit so as to be used as the carbon source of the medium. In a case where the rest of the pretreated biomass is used as the carbon source, a part of the pretreated biomass is sent to the saccharification device by using a first treatment means.

However, in the pretreated biomass, the inhibitant may be contained, which inhibits growth and propagation of the enzyme-producing microorganism. The inhibitant includes for example formic acid and acetic acid. As described above, by controlling the operation condition of the pretreatment device or the property of the biomass before the pretreatment, the amount of the inhibitant can be decreased, as a result, inhibition of growth of the enzyme-producing microorganism is prevented, therefore, the high yield of the saccharifying enzyme can be stably obtained. The preferable values of the pH, the acidity, the alkalinity, the ultraviolet absorbance, the amount of the residual solid, and the amount of the decomposition product are similar as those stated before and the controlling in the pretreatment is also performed in the same way as stated before.

Although the fermentation temperature and time for the propagating differ depending on the strain of the enzyme-producing microorganism, the propagation is normally performed at the temperature of 28 to 32° C. for 48 hours to 10 days.

Further, a fermentation vessel used for the propagation in the saccharifying enzyme production device includes for example an aerated mixing vessel, a bubble column type vessel, a fluidized bed type vessel, and a packed column type vessel.

By the propagation of the enzyme-producing microorganism, the enzyme-producing microorganism is grown and propagated, and further the enzyme-producing microorganism produces the saccharifying enzyme. A liquid culture for the propagation of the enzyme-producing microorganism, at the end of the propagation, contains the saccharifying enzyme. The enzyme-containing liquid maybe supplied to the saccharification device directly, or supplied to the saccharification device after purification.

In a general method of enzyme purification, at first, the enzyme-producing microorganism is removed by filtration, centrifugation, or the like so as to obtain a supernatant. Further, from the supernatant, the saccharifying enzyme as the target is obtained through chromatography such as ion exchange chromatography, isoelectric chromatography, gel filtration chromatography, hydrophobic chromatography, adsorption column chromatography, affinity chromatography, and reverse phase column chromatography; precipitation separation using ethanol, methanol, acetone, or the like; and a filtration treatment such as microfiltration, ultrafiltration, and reverse osmosis filtration.

### EXAMPLES

Bagasse was used as a raw material of biomass, and subjected to a pretreatment by a hydrothermal treatment followed by simultaneous saccharification and fermentation so as to produce ethanol.

The bagasse was washed with water to remove foreign materials. Then a pressure vessel was filled with the bagasse, was heated to 180 to 220° C. by injecting saturated steam at 25 bar into the pressure vessel and subjected to the hydrothermal treatment for 4 to 10 minutes as the pretreatment. The thus obtained hydrothermal treatment product was subjected to the simultaneous saccharification and fermentation with the cellulase enzyme, which had been produced by the Acremonium cellulolyticus C-1 strain (Ferm P-18058), and with the yeast of Saccharomyces cerevisiae. The simultaneous saccharification and fermentation was performed at 35° C. for 48 hours where the hydrothermal treatment product was added so as to have the concentration of 10 dry weight % of the total amount of the fermentation solution, the cellulase was added as the enzyme in the amount of 10 FPU/g-solid and the propagation broth of yeast was added in the amount of 5% of the total amount of the fermentation broth.

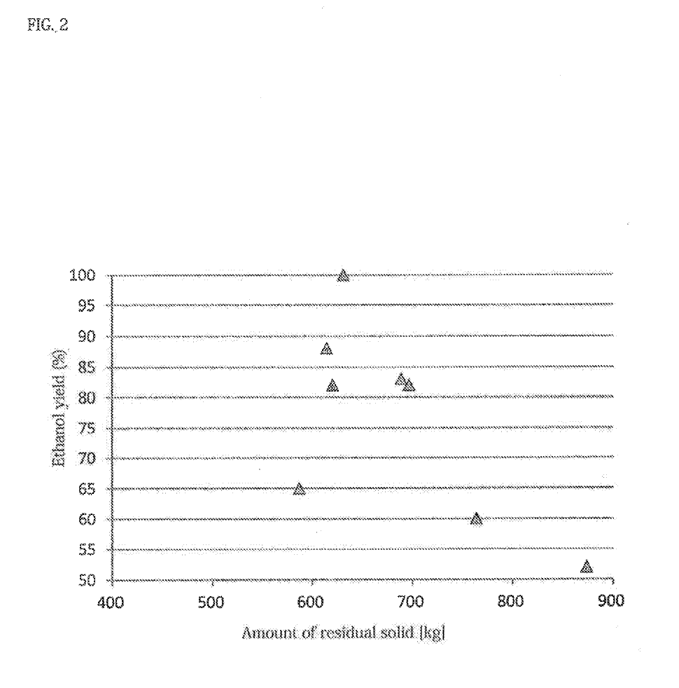

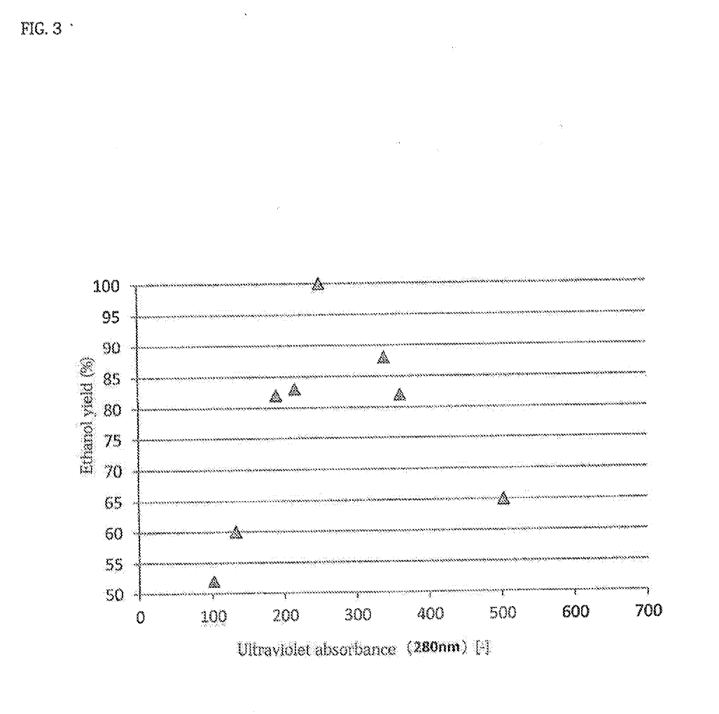

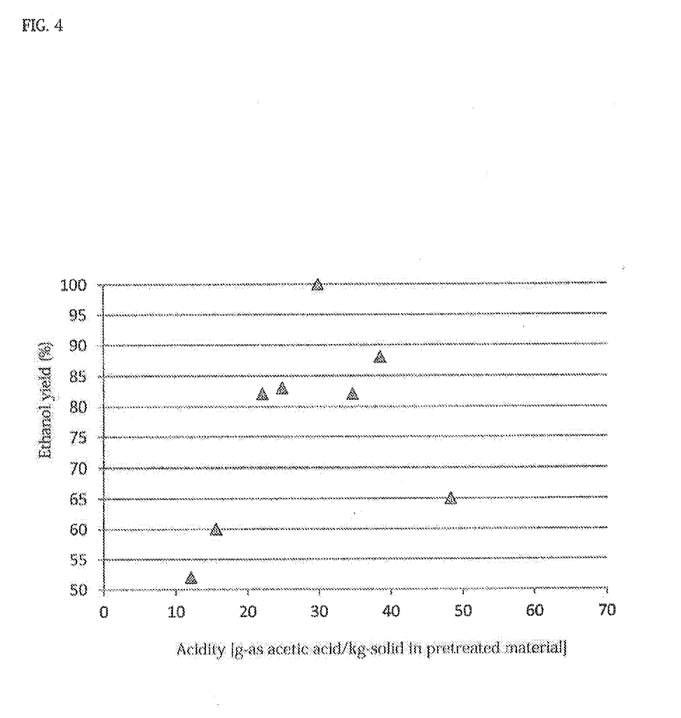

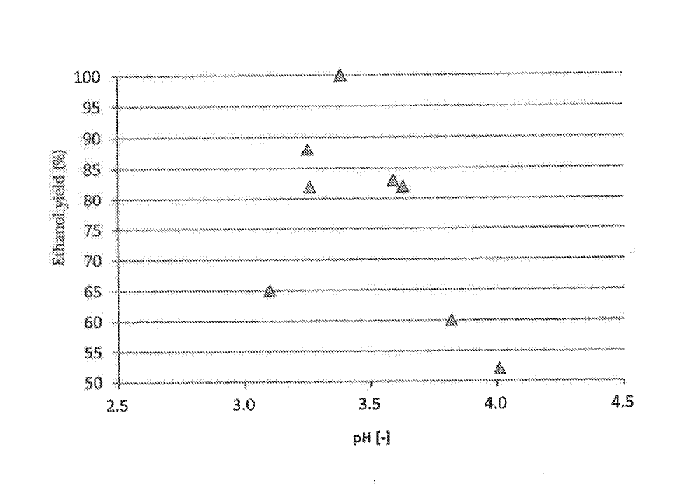

The results are shown in FIGS. 2 to 5.

Provided that the maximum yield of ethanol is 100%, the 80% or more yield of the ethanol can be achieved when the amount of the residual solid was 600 to 750 kg, the ultraviolet absorbance was 180 to 400, the acidity was 20 to 40 g/kg-solid, and the pH was 3.2 to 3.8. That is, when the amount of the residual solid is 600 to 750 kg for 1,000 kg of the pretreated biomass, when the ultraviolet absorbance is 180 to 400 when the acidity is 20 to 40 g/kg-solid, or when the pH is 3.2 to 3.8, the high yield of the ethanol can be stably obtained.

1. A biomass treatment system, comprising:
a pretreatment device pretreating a biomass;
a measurement unit taking at least one measurement selected from the group consisting of pH, acidity, alkalinity, ultraviolet absorbance, an amount of a decomposition product, and an amount of an undecomposed substance of the pretreated biomass; and
a control unit controlling an operation condition of the pretreatment device or property of the biomass before the pretreatment on the basis of a measurement result obtained by the measurement unit.

2. The biomass treatment system according to claim 1,
wherein the control unit controls at least one of a reaction time of the pretreatment, a reaction temperature of the pretreatment, pH of the biomass before the pretreatment, acidity of the biomass before the pretreatment, and alkalinity of the biomass before the pretreatment.

3. The biomass treatment system according to claim 1, further comprising:
a saccharification and fermentation unit in which the biomass pretreated under controlling by the control unit is saccharified with a saccharifying enzyme, and the saccharified biomass is fermented with a fermentive microorganism.

4. The biomass treatment system according to claim 1, further comprising:
a saccharification and fermentation unit in which the biomass pretreated under controlling by the control unit is saccharified with a saccharifying enzyme, and the saccharified biomass is fermented with a fermentive microorganism;
a saccharifying enzyme production device producing the saccharifying enzyme to be used in the saccharification and fermentation unit;
a first treatment unit sending a part of the pretreated biomass without any treatment to the saccharification and fermentation unit; and
a second treatment unit feeding a rest of the pretreated biomass to the saccharifying enzyme production device, propagating a microorganism which produces saccharifying enzyme by using the fed pretreated biomass as a medium so as to produce the saccharifying enzyme, and sending the produced saccharifying enzyme to the saccharification and fermentation unit.

5. A method for treating a biomass, comprising:
a step of pretreating the biomass by a pretreatment device;
a step of taking at least one measurement, the measurement selected from the group consisting of pH, acidity, alkalinity, ultraviolet absorbance, an amount of a decomposition product, and an amount of an undecomposed substance of the pretreated biomass by a measurement unit; and
a step of controlling an operation condition of the pretreatment device or a property of the biomass before the pretreatment on the basis of a measurement result obtained by the measurement unit.
